(19) United States
(12) Patent Application Publication
(10) Pub. No.: US 2022/0000348 A1
(43) Pub. Date: Jan. 6, 2022

(54) GAS FEEDING APPARATUS, GAS FEEDING AMOUNT CONTROL METHOD AND GAS FEEDING SYSTEM

(71) Applicant: OLYMPUS CORPORATION, Tokyo (JP)

(72) Inventors: Goki NUMATA, Tokyo (JP); Kunitoshi HIRAGA, Tokyo (JP)

(73) Assignee: OLYMPUS CORPORATION, Tokyo (JP)

(21) Appl. No.: 17/476,714

(22) Filed: Sep. 16, 2021

Related U.S. Application Data
(63) Continuation of application No. PCT/JP2019/011791, filed on Mar. 20, 2019.

(51) Int. Cl. A61B 1/015; A61B 1/00

(52) U.S. Cl. CPC A61B 1/015; A61B 2562/0247; A61B 1/00068

(57) ABSTRACT

A gas feeding apparatus communicating with a gas feeding source that feeds a predetermined gas and configured to supply the gas to a lumen of a patient through a gas feeding conduit provided in an endoscope includes: a gas feeding bottle provided between the gas feeding apparatus and the gas feeding conduit of the endoscope, and having the gas reserved; and a control circuit configured to detect a timing of start of decrease in a state where a predetermined value is kept, and configured to control a gas feeding amount from the gas feeding bottle to the gas feeding conduit, at the detected timing.

### CROSS REFERENCE TO RELATED APPLICATION

This application is a continuation application of PCT/JP2019/011791 filed on Mar. 20, 2019, the entire contents of which are incorporated herein by this reference.

### BACKGROUND OF THE INVENTION

### 1. Field of the Invention

An embodiment of the present invention relates to a gas feeding apparatus, a gas feeding amount control method and a gas feeding system, and particularly relates to a gas feeding apparatus, a gas feeding amount control method and a gas feeding system by which air or an inactive gas is injected from a distal end of an endoscope apparatus into an abdominal cavity or a lumen while the gas feeding apparatus is connected with the endoscope apparatus.

### 2. Description of the Related Art

In recent years, an endoscope has been widely used. The endoscope is used for observing organs and the like in a lumen of a patient and performing various curative treatments to sites and tissues in the lumen using treatment instruments as necessary. For the observation and various treatments using the endoscope, a gas feeding apparatus is used for purpose of securing a viewing field of the endoscope and purpose of securing a region for operating treatment instruments.

The gas feeding apparatus injects carbon dioxide gas or the like to an operative field in a body cavity, as a gas for gas feeding, and secures the viewing field and of the endoscope and the operation region for treatment instruments (see Japanese Patent Application Laid-Open Publication No. 2016-144579, for example).

### SUMMARY OF THE INVENTION

A gas feeding apparatus in an aspect of the present invention is a gas feeding apparatus communicating with a gas feeding source that feeds a predetermined gas, and configured to supply the predetermined gas to a lumen of a patient through a gas feeding conduit provided in an endoscope, the gas feeding apparatus including: a pressure sensor configured to measure a pressure in a gas feeding bottle provided between the gas feeding apparatus and the gas feeding conduit of the endoscope, the predetermined gas being reserved in the gas feeding bottle; and a control circuit configured to detect a timing when a measurement result of the pressure sensor starts to decrease in a state where the measurement result of the pressure sensor is kept at a predetermined value, and configured to control a gas feeding amount from the gas feeding bottle to the gas feeding conduit, at the detected timing.

A gas feeding amount control method in an aspect of the present invention is a gas feeding amount control method for a gas feeding apparatus configured to supply a predetermined gas, the gas feeding amount control method including: measuring a pressure in a gas feeding bottle in which the predetermined gas is reserved; detecting a timing when a measurement result of the pressure starts to decrease in a state where the measurement result of the pressure sensor is kept at a predetermined value; and controlling a gas feeding amount from the gas feeding bottle to a gas feeding conduit, based on the detected timing.

A gas feeding system in an aspect of the present invention is a gas feeding system including a gas feeding apparatus communicating with a gas feeding source that feeds a predetermined gas, and configured to supply the predetermined gas to a lumen of a patient through a gas feeding conduit provided in an endoscope, and a gas feeding bottle provided between the gas feeding apparatus and the gas feeding conduit of the endoscope, the predetermined gas being reserved in the gas feeding bottle, the gas feeding apparatus including: a pressure sensor configured to measure a pressure in the gas feeding bottle provided between the gas feeding apparatus and the gas feeding conduit of the endoscope, the predetermined gas being reserved in the gas feeding bottle; and a control circuit configured to detect a timing when a measurement result of the pressure of the pressure sensor starts to decrease in a state where the measurement result of the pressure of the pressure sensor is kept at a predetermined value, and configured to control a gas feeding amount from the gas feeding bottle to the gas feeding conduit, at the detected timing.

### BRIEF DESCRIPTION OF THE DRAWINGS

FIG. 1 is a diagram for describing an example of a whole configuration of a gas feeding system according to a first embodiment of the present invention;

FIG. 2 is a diagram showing an example of temporal change in gas feeding bottle internal pressure;

FIG. 3 is a diagram showing an example of temporal change in gas feeding flow rate;

FIG. 4 is a diagram for describing an example of a whole configuration of a gas feeding system according to a second embodiment;

FIG. 5 is a diagram for describing an example of a relation between the gas feeding flow rate and a lumen internal pressure; and

FIG. 6 is a diagram for describing an example of a whole configuration of a gas feeding system according to a modification of the second embodiment.

### DETAILED DESCRIPTION OF THE PREFERRED EMBODIMENTS

Embodiments will be described below with reference to the drawings.

### First Embodiment

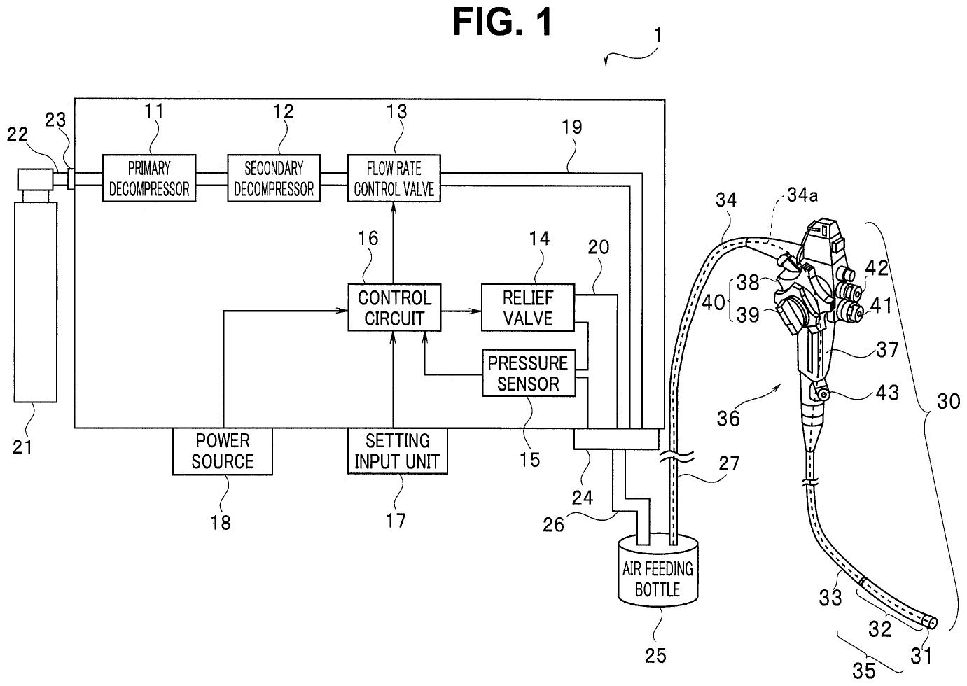

FIG. 1 is a diagram for describing an example of a whole configuration of a gas feeding system according to a first embodiment of the present invention. As shown in FIG. 1, the gas feeding system in the present embodiment is configured by a gas feeding apparatus 1 and a gas feeding bottle 25.

The gas feeding apparatus 1 is connected with a gas supply source 21 (for example, a carbon dioxide gas cylinder) through a high-pressure gas hose 22. The other end of the high-pressure gas hose 22 is connected with a high-pressure connector 23 provided on the gas feeding apparatus 1. Further, a gas feeding connector 24 is provided on the gas feeding apparatus 1, and one end of a gas feeding tube 26 is connected with the gas feeding connector 24. The other end of the gas feeding tube 26 is connected with a gas feeding bottle 25 as a gas reserve unit. One end of a gas feeding tube 27 is connected with the gas feeding bottle 25. The other end of the gas feeding tube 27 is connected with an unillustrated gas feeding conduit through an unillustrated endoscope connector. The gas feeding conduit is inserted in a universal cable 34 connected with an endoscope 30.

The endoscope 30 is configured to include an insertion unit 35 having a long size and an elongated shape, an operation unit 36, and the universal cable 34. The insertion unit 35 of the endoscope 30 is configured to include a distal end portion 31, a bending portion 32 and a flexible tube portion 33, in an order from a distal end. At the distal end portion 31, an objective lens (not illustrated) that forms an image of a subject is disposed. Furthermore, at an image formation position of the objective lens, a solid-state image pickup device (not illustrated) such as a CCD and a CMOS is disposed as image pickup means.

On the operation unit 36, a bending operation knob 40 for performing a bending operation of the bending portion 32 of the insertion unit 35 is arranged in a rotatable manner, and switches for various endoscope functions, and the like are provided. Note that a UD bending operation knob 38 and an RL bending operation knob 39 are arranged so as to be superimposed on each other in the bending operation knob 40. By the UD bending operation knob 38, the bending operation of the bending portion 32 is performed in an up-down direction, and by the RL bending operation knob 39, the bending operation of the bending portion 32 is performed in a right-left direction. Furthermore, a gas feeding button 41 for supplying a gas such as carbon dioxide into the lumen, and a suction button 42 are provided on the operation unit 36.

A joining portion between the insertion unit 35 and the operation unit 36 is configured to include a grasping portion 37 and a treatment instrument channel insertion portion 43. The grasping portion 37 also serves as a grasping portion for a user. The treatment instrument channel insertion portion 43 is arranged on a bend preventing portion provided between the grasping portion 37 and one end of the flexible tube portion 33 of the insertion unit 35, and serves as an opening portion of a treatment instrument channel into which various treatment instruments arranged on the insertion unit 35 are inserted.

The universal cable 34 extended from the operation unit 36 includes an unillustrated endoscope connector at an extension end. The gas feeding tube 27 extended from the gas feeding bottle 25 is connected with a gas feeding conduit 34a (shown by a dotted line) inserted in the universal cable 34, through the endoscope connector. In other words, the gas discharged from the gas feeding apparatus 1 is supplied to the gas feeding conduit 34a, through the gas feeding tube 26, the gas feeding bottle 25 and the gas feeding tube 27. The gas supplied to the gas feeding conduit 34a is supplied from the distal end portion 31 of the insertion unit 35 into the lumen, when a gas feeding button 41 of the operation unit 36 is pressed down.

In the gas feeding apparatus 1, a primary decompressor 11, a secondary decompressor 12 and a flow rate control valve 13 are provided, and the sites are connected in this order, by a gas feeding conduit 19 formed of silicon, fluorine resin or the like. Further, a relief valve 14 and a pressure sensor 15 also are provided in the gas feeding apparatus 1, and the sites are connected with an exhaust conduit 20. Further, a control circuit 16, a setting input unit 17 and a power source 18 also are provided in the gas feeding apparatus 1.

The primary decompressor 11 and the high-pressure connector 23 are connected by the gas feeding conduit 19, and the flow rate control valve 13 and the gas feeding connector 24 are connected by the gas feeding conduit 19. In other words, the gas fed from the gas supply source 21 and supplied to the gas feeding apparatus 1 through the high-pressure gas hose 22 passes through the primary decompressor 11, the secondary decompressor 12 and the flow rate control valve 13 in this order, along the gas feeding conduit 19. After adjustment to a predetermined pressure and flow rate, the gas is discharged from the gas feeding tube 26 through the gas feeding connector 24.

The primary decompressor 11 and the secondary decompressor 12 decompress the gas such as carbon dioxide, which is supplied through the high-pressure connector 23, to a pressure that is not dangerous to human bodies. For example, the primary decompressor 11 and the secondary decompressor 12 decompress the gas supplied from the gas supply source 21 at a high pressure of about 1 MPa, to about 6 kPa to 50 kPa.

The flow rate control valve 13 is configured to be capable of adjusting the flow rate of the gas to be supplied to the endoscope 30, to a predetermined value. For example, the flow rate control valve 13 is a kind of an electromagnetic drive valve, and is configured by a control valve in which an electromagnetic coil is used as a drive unit. When electric current is caused to flow through the electromagnetic coil, magnetic force is generated, and a plunger is pulled, so that the valve is opened or closed. The position of the plunger is controlled by the magnitude of the electric current that is caused to flow through the electromagnetic coil, and thereby, an opening degree of a valve unit is controlled, so that the flow rate of the gas that flows through the gas feeding conduit can be adjusted to a predetermined value.

The relief valve 14, which is an opening-closing valve, is connected with the gas feeding connector 26 through the exhaust conduit 20. The relief valve 14 is basically closed both during the action of the gas feeding and during the stop of the gas feeding, and when it is necessary to release the gas in the gas feeding bottle 25, an opening-closing action is performed based on a control signal inputted from the control circuit 16. In other words, when the relief valve 14 is opened, the gas reserved in the gas feeding bottle 25 is released to the atmosphere, through the gas feeding tube 26, the gas feeding connector 24, the exhaust conduit 20 and the relief valve 14.

Note that it is preferable that a resistance of the exhaust conduit 20 should be lower than a resistance of the gas feeding conduit inserted in the insertion unit of the endoscope, in order to quickly emit the gas reserved in the gas feeding bottle 25. For example, the exhaust conduit 20 is configured by a conduit having a larger diameter than the gas feeding conduit of the endoscope, and thereby, can be configured as a conduit having a lower resistance. In the case where the resistance value is adjusted by conduit diameter, for example, when the gas feeding conduit of the endoscope has a diameter of about 1 mm, the exhaust conduit 20 can be configured by a conduit having a diameter of about 1 cm to 2 cm.

The pressure sensor 15 as a pressure measurement unit measures a pressure in the gas feeding bottle 25, through the exhaust conduit 20, the gas feeding connector 24 and the gas feeding tube 26. The measurement of the pressure is performed during the stop of the gas feeding. A measurement result of the pressure sensor 15 is outputted to the control circuit 16.

The setting input unit 17 is a user interface through which the user or the like performs setting of a gas feeding flow rate or inputs an instruction of gas feeding start or gas feeding stop. Note that the setting input unit 17 may be configured such that a flow rate setting unit to perform the setting of the gas feeding flow rate and a gas feeding switch to perform the instruction of the gas feeding start or gas feeding stop are separated. The instruction content from the setting input unit 17 is outputted to the control circuit 16.

The power source 18 switches on-off of power supply to each site of the gas feeding apparatus 1.

The control circuit 16 as a control unit gives, to the flow rate control valve 13, an instruction of the opening-closing action or the opening degree of the valve that corresponds to the setting flow rate, in accordance with the instruction from the setting input unit 17. Further, the control circuit 16 gives the instruction of the opening-closing action to the relief valve 14, based on a pressure measurement value inputted from the pressure sensor 15.

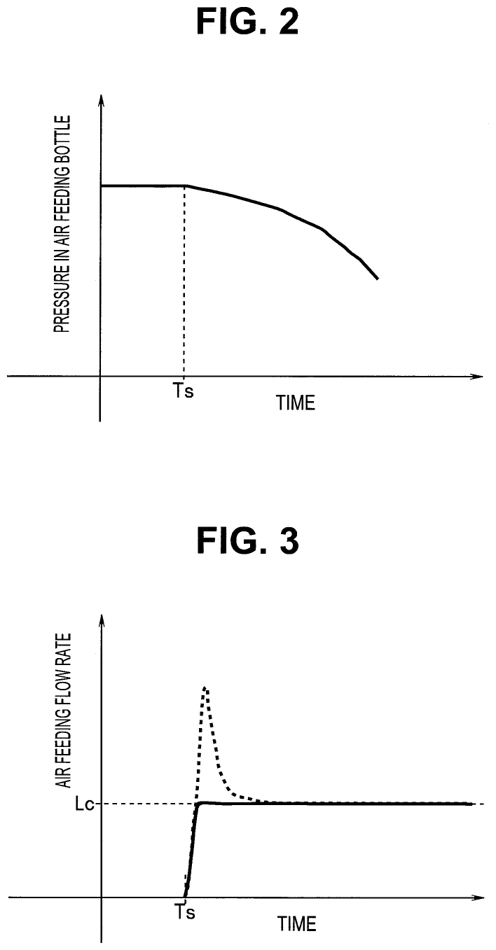

Next, the opening-closing action of the relief valve 14 based on the instruction from the control circuit 16 will be described. FIG. 2 is a diagram showing an example of temporal change in gas feeding bottle internal pressure. In other words, FIG. 2 shows the temporal change in the measurement value of the pressure sensor 15.

In a state where the gas feeding is stopped by the gas feeding button 41 of the endoscope 30, the pressure in the gas feeding bottle 25 is a gas feeding pressure decompressed by the secondary decompressor 12, without depending on the set gas feeding flow rate. In other words, the pressure of the gas reserved in the gas feeding bottle 25 is a higher pressure than the gas feeding pressure during the gas feeding. In this state, when the gas feeding from the gas feeding apparatus 1 to the lumen through the endoscope 30 is started by the operation of the gas feeding button 41, the gas reserved in the gas feeding bottle 25 is emitted from the distal end portion 31 of the endoscope 30 to the lumen, and therefore, the pressure in the gas feeding bottle 25 decreases. In other words, it can be estimated that the gas feeding was started at a timing Ts when the pressure in the gas feeding bottle stated to decrease from a predetermined value in FIG. 2.

The control circuit 16 monitors the measurement value inputted from the pressure sensor 15, and controls and opens the relief valve 14 when detecting the start of the decrease in the pressure in the gas feeding bottle 25. Since the relief valve 14 switches to the opening state, the gas reserved in the gas feeding bottle 25 is released to the atmosphere through the gas feeding tube 26, the gas feeding connector 24, the exhaust conduit 20 and the relief valve 14, so that the pressure in the gas feeding bottle 25 decreases. After a preset time period from the switching of the relief valve 14 to the opening state, the control circuit 16 switches the relief valve 14 to the closing state. While the relief valve 14 is in the opening state, the gas feeding to the endoscope 30 is hardly performed, and the gas to be fed is released to the atmosphere. Accordingly, it is desirable to switch the relief valve 14 to the closing state and return to a normal gas feeding state without delay, when the gas reserved in the gas feeding bottle 25 has been released to the atmosphere after the switching of the relief valve 14 to the opening state.

FIG. 3 is a diagram showing an example of temporal change in gas feeding flow rate. In the figure, the temporal change in gas feeding flow rate in the case where the opening-closing action of the relief valve 14 is performed just after the start of the gas feeding is shown by a solid line. Further, the temporal change in gas feeding flow rate in the case where the opening-closing action of the relief valve 14 is not performed is shown by a dotted line. Before the timing Ts when the gas feeding is started by the gas feeding button 41, the gas feeding is stopped, and therefore, the gas feeding flow rate is zero.

In the case where the opening-closing action of the relief valve 14 is not performed just after the timing Ts of the start of the gas feeding, the gas reserved in the gas feeding bottle 25 is fed to the gas feeding tube 27 at once, and therefore, the gas feeding flow rate becomes higher than a setting flow rate Lc. The gas fed at a high flow rate is released to the lumen from the distal end portion 31 of the endoscope 30, and therefore, gets to have a setting flow rate Lc in a short time. However, due to the excessive gas feeding just after the timing Ts of the start of the gas feeding, the lumen expands more than necessary, so that peristatic motion of an affected part can occur. Generally, in the case where the gas feeding into the lumen is performed by the gas feeding apparatus 1, the setting flow rate Lc has a low value (about 0.9 L/min), for minutely adjusting the distance between an organ in the lumen and the distal end portion 30. Accordingly, even a slight fluctuation of the gas feeding flow rate can increase the distance between the distal end portion 30 and the affected part. There is a problem in that the treatment of the affected part becomes difficult due to the peristatic motion of the affected part and the increase in the distance between the distal end portion 30 and the affected part.

On the other hand, in the case where the opening-closing action of the relief valve 14 is performed just after the timing Ts of the start of the gas feeding, the gas reserved in the gas feeding bottle 25 can be released from the relief valve 14 to the atmosphere just after the start of the gas feeding, and therefore, it is possible to prevent overshoot of the gas feeding flow rate, and to stably adjust the gas feeding flow rate to the distal end portion 30, to the setting flow rate Lc, from the time just after the start of the gas feeding. Accordingly, it is possible to prevent the excessive gas feeding to the lumen.

In this way, according to the present embodiment, the pressure sensor 15 configured to measure the pressure in the gas feeding bottle 25 is provided, and the control circuit 16 configured to detect the timing of the switching from the stop of the gas feeding to the start of the gas feeding based on the measurement value of the pressure sensor 15 and configured to control the gas feeding amount to the endoscope 30 is provided. By releasing the gas reserved in the gas feeding bottle 25 to the atmosphere just after the start of the gas feeding, it is possible to prevent the overshoot of the gas feeding flow rate just after the start of the gas feeding, and to prevent the excessive gas feeding to the lumen.

Note that although the configuration of the system that can perform the gas feeding to the lumen has been described above as an example, the present embodiment can be applied to a gas/water feeding system that can perform not only gas feeding but also water feeding. The gas/water feeding system uses a water feeding bottle as the gas feeding bottle 25, and uses a gas/water feeding button that allows switching and execution of the gas feeding and the water feeding, as the gas feeding button 41.

### Second Embodiment

The gas feeding apparatus 1 of the gas feeding system in the first embodiment described above opens the relief valve 14 at the timing when the gas feeding into the lumen is started, and thereby, decreases the pressure in the gas feeding bottle 25, so that the overshoot of the gas feeding flow rate just after the start of the gas feeding is restrained. In contrast, the present embodiment is different in that a pressure control valve 51 is provided and the pressure of the gas feeding gas is adjusted depending on the setting value of the gas feeding flow rate, so that the overshoot of the gas feeding flow rate just after the start of the gas feeding is restrained.

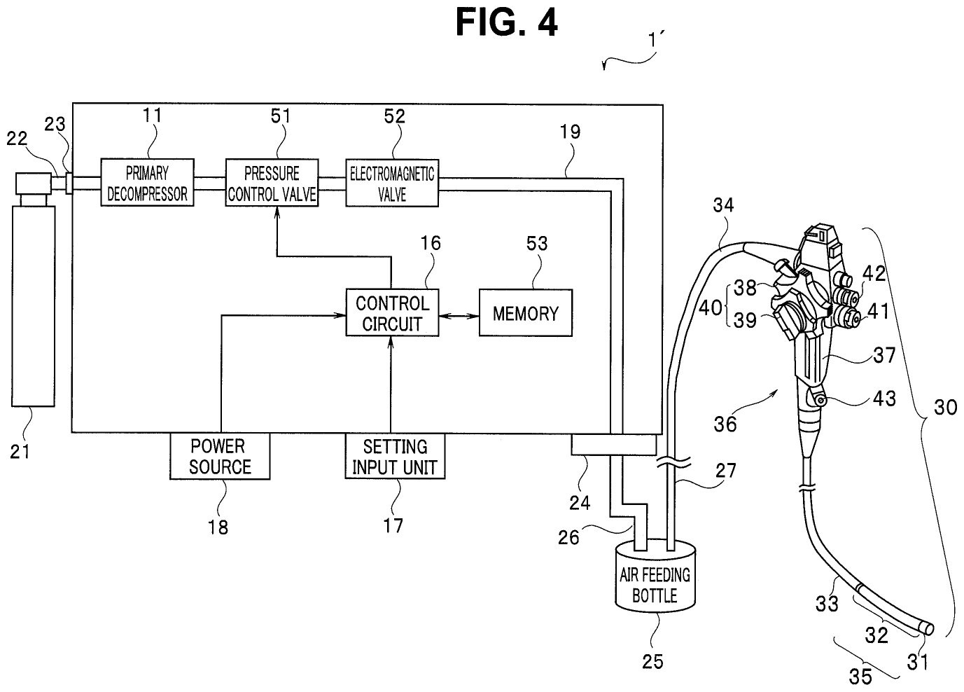

A configuration of a gas feeding system in the present embodiment will be described below with use of FIG. 4. FIG. 4 is a diagram for describing an example of a whole configuration of the gas feeding system according to the second embodiment. Note that component elements other than a gas feeding apparatus 1′ are the same as component elements in the first embodiment, in the gas feeding system in the present embodiment. Further, in the gas feeding apparatus 1′, component elements other than the pressure control valve 51, an electromagnetic valve 52 and a memory 53 are the same as component elements in the first embodiment. The same component elements are denoted by the same reference characters, and detailed descriptions are omitted. Further, the gas feeding apparatus 1′ is not provided with the flow rate control valve 13, the relief valve 14 and the pressure sensor 15, which are included in the gas feeding apparatus 1.

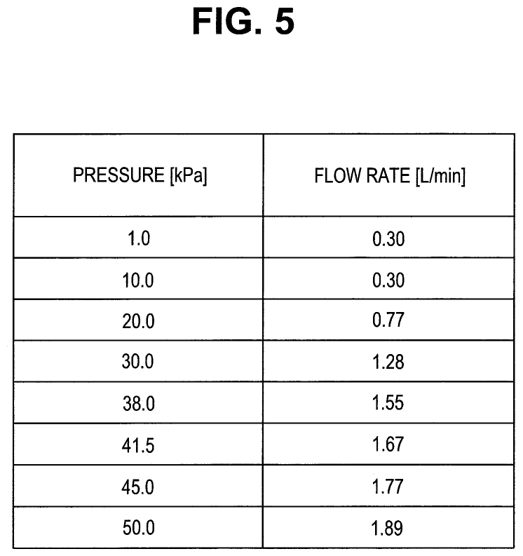

The pressure control valve 51 is a control value that can adjust the pressure (referred to as a conduit internal pressure, hereinafter) of the gas that flows through the gas feeding conduit 19. For example, the pressure control valve 51 is configured so as to electrically adjust the conduit internal pressure such that the conduit internal pressure becomes a predetermined value by changing force of a decompression spring that acts on a valve unit. The electromagnetic valve 52 performs an opening-closing action based on a control signal that is inputted from the control unit 24. In the memory 53, a relation between the setting flow rate of the gas feeding gas and the conduit internal pressure is previously registered. FIG. 5 is a diagram for describing an example of the relation between the gas feeding flow rate and the conduit internal pressure.

For example, before use for an actual procedure, a gas feeding action test is performed in advance using the gas feeding apparatus 1′, and a table shown in FIG. 5 is generated based on data acquired by actually measuring the gas feeding flow rate and the conduit internal pressure. The relation between the gas feeding flow rate and the conduit internal pressure that is previously registered in the memory 53 does not need to be based on actual measurement values, and the relation between the two may be acquired and registered by another technique such as a simulation or design values of the apparatus. Further, in the memory 53, the relation between the gas feeding flow rate and the conduit internal pressure may be registered in a format of a correspondence table described above, or may be registered in a format of an equation indicating a correlation between the gas feeding flow rate and the conduit internal pressure.

The control circuit 16 calculates a setting value of the conduit internal pressure by comparing the setting value of the gas feeding flow rate that is inputted from the setting input unit 17, with the correspondence relation data of the gas feeding flow rate and the conduit internal pressure that is registered in the memory 53. For example, in the case where the gas feeding flow rate is set to 1.55 L/min, the setting value of the conduit internal pressure is 38.0 kPa. The control circuit 16 controls the action of the pressure control valve 51, based on the calculated setting value of the conduit internal pressure.

In this way, according to the present embodiment, the pressure control valve 51 is provided in the middle of the gas feeding conduit 19, and the pressure control valve 51 is controlled such that a conduit internal pressure corresponding to the setting value of the gas feeding flow rate is kept. Accordingly, the conduit internal pressure is constantly controlled to a value corresponding to the gas feeding flow rate, and therefore, it is possible to prevent the overshoot of the gas feeding flow rate just after the start of the gas feeding, and to prevent the excessive gas feeding to the lumen.

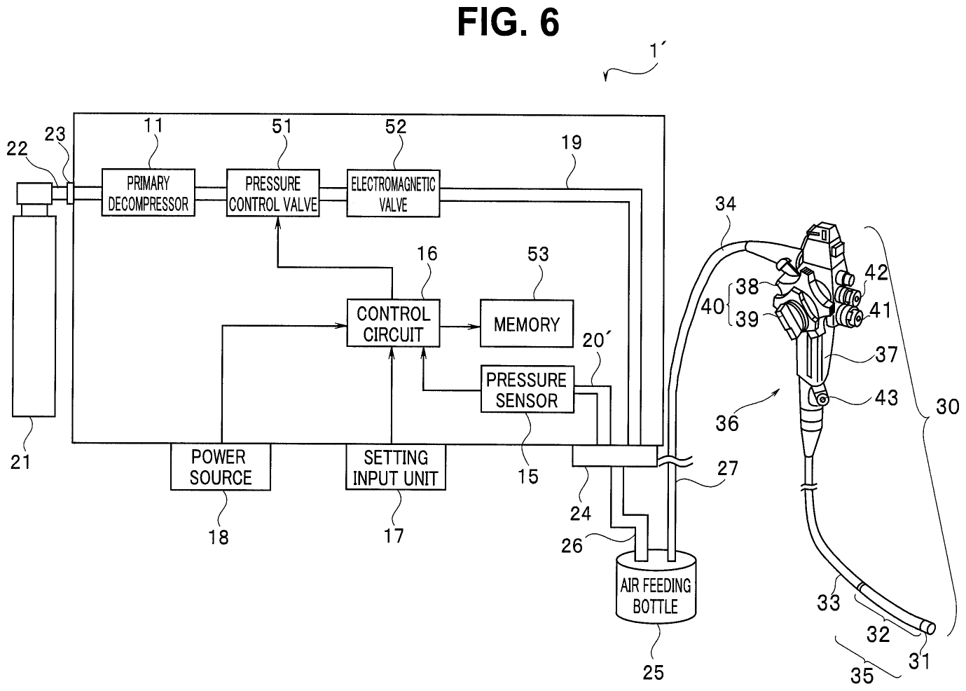

FIG. 6 is a diagram for describing an example of a whole configuration of a gas feeding system according to a modification of the second embodiment. As shown in FIG. 6, the gas feeding apparatus 1′ in the second embodiment may be configured to monitor the pressure in the gas feeding bottle 25 through an exhaust conduit 20′, using the pressure sensor 15 provided in the gas feeding apparatus 1 in the first embodiment. The control circuit 16 adjusts a control value of the pressure control valve 51 in response to a fluctuation of the measurement value of the pressure sensor 15, while controlling the pressure control valve 51 by calculating the setting value of the conduit internal pressure based on the gas feeding flow rate. Thereby, it is possible to further stably control the conduit internal pressure, and to further enhance the effect of the prevention of the overshoot of the gas feeding flow rate just after the start of the gas feeding. Accordingly, it is possible to prevent the excessive gas feeding to the lumen.

Although some embodiments of the present invention have been described, the embodiments have been shown as examples, and are not intended to limit the scope of the invention. The novel embodiments can be carried out in a variety of other modes, and various omissions, replacements and alterations can be performed without departing from the spirit of the invention. The embodiments and the modifications are included in the scope and spirit of the invention, and are included in a scope equivalent to the invention described in the claims.

What is claimed is:

1. A gas feeding apparatus communicating with a gas feeding source that feeds a predetermined gas, and configured to supply the predetermined gas to a lumen of a patient through a gas feeding conduit provided in an endoscope, the gas feeding apparatus comprising:
a pressure sensor configured to measure a pressure in a gas feeding bottle provided between the gas feeding apparatus and the gas feeding conduit of the endoscope, the predetermined gas being reserved in the gas feeding bottle; and
a control circuit configured to detect a timing when a measurement result of the pressure sensor starts to decrease in a state where the measurement result of the pressure sensor is kept at a predetermined value, and configured to control a gas feeding amount from the gas feeding bottle to the gas feeding conduit, at the detected timing.

2. The gas feeding apparatus according to claim 1, wherein
the gas feeding apparatus further comprises an exhaust conduit connected with the gas feeding bottle, and an open valve configured to open and close the exhaust conduit, and
the control circuit is configured to control an opening-closing action of the open valve based on the measurement result of the pressure sensor.

3. The gas feeding apparatus according to claim 2, wherein
an inner diameter of the exhaust conduit is larger than an inner diameter of the gas feeding conduit.

4. A gas feeding amount control method for a gas feeding apparatus configured to supply a predetermined gas, the gas feeding amount control method comprising:
measuring a pressure in a gas feeding bottle in which the predetermined gas is reserved;
detecting a timing when a measurement result of the pressure starts to decrease in a state where the measurement result of the pressure is kept at a predetermined value; and
controlling a gas feeding amount from the gas feeding bottle to a gas feeding conduit, based on the detected timing.

5. A gas feeding system including:
a gas feeding apparatus communicating with a gas feeding source that feeds a predetermined gas, and configured to supply the predetermined gas to a lumen of a patient through a gas feeding conduit provided in an endoscope; and
a gas feeding bottle provided between the gas feeding apparatus and the gas feeding conduit of the endoscope, the predetermined gas being reserved in the gas feeding bottle,
the gas feeding apparatus comprising:
a pressure sensor configured to measure a pressure in the gas feeding bottle provided between the gas feeding apparatus and the gas feeding conduit of the endoscope, the predetermined gas being reserved in the gas feeding bottle; and
a control circuit configured to detect a timing when a measurement result of the pressure of the pressure sensor starts to decrease in a state where the measurement result of the pressure of the pressure sensor is kept at a predetermined value, and configured to control a gas feeding amount from the gas feeding bottle to the gas feeding conduit, at the detected timing.

6. The gas feeding system according to claim 5, wherein
the gas feeding system further comprises an exhaust conduit connected with the gas feeding bottle, an open valve configured to open and close the exhaust conduit, and the endoscope connected with the gas feeding bottle through the gas feeding conduit, and
the control circuit is configured to control an opening-closing action of the open valve based on the measurement result of the pressure sensor.
